(19) United States
(12) Patent Application Publication
(10) Pub. No.: US 2023/0054013 A1
(43) Pub. Date: Feb. 23, 2023

(54) System and Method for Adaptive Auditory Wellness

(71) Applicant: Dell Products L.P., Round Rock, TX (US)

(72) Inventors: Harpreet S. Narula, Austin, TX (US); Vivek Viswanathan Iyer, Austin, TX (US)

(73) Assignee: Dell Products L.P., Round Rock, TX (US)

(21) Appl. No.: 17/407,211

(22) Filed: Aug. 20, 2021

(51) Int. Cl. G16H 50/30; G16H 40/20; G06F 3/16

(52) U.S. Cl. CPC G16H 50/30; G16H 40/20; G06F 3/165

(57) ABSTRACT

A system, method, and computer-readable medium are disclosed for auditory adaptive wellness. A user's sensitivity to noise and frequency is calibrated. Thresholds are set for three use cases of dosage fatigue, background cumulative frequency and general frequency fatigue. Audio cumulation rates of peripherals for is determined for the three use cases. Cumulative dosage exposure (CDE), cumulative background exposure (CBE) and cumulative frequency exposure (CFE) are calculated. Warnings and recommendations are provided when CDE exceeds the threshold for dosage fatigue, CBE exceeds the threshold for background cumulative frequency fatigue, and CFE exceeds the threshold for general frequency fatigue.

### BACKGROUND OF THE INVENTION

### Field of the Invention

The present invention relates to the management of information handling systems. More specifically, embodiments of the invention provide a system, method, and computer-readable medium for adaptive auditory wellness for users of information handling systems.

### Description of the Related Art

As the value and use of information continues to increase, individuals and businesses seek additional ways to process and store information. One option available to users is information handling systems. An information handling system generally processes, compiles, stores, and/or communicates information or data for business, personal, or other purposes thereby allowing users to take advantage of the value of the information. Because technology and information handling needs and requirements vary between different users or applications, information handling systems may also vary regarding what information is handled, how the information is handled, how much information is processed, stored, or communicated, and how quickly and efficiently the information may be processed, stored, or communicated. The variations in information handling systems allow for information handling systems to be general or configured for a specific user or specific use such as financial transaction processing, airline reservations, enterprise data storage, or global communications. In addition, information handling systems may include a variety of hardware and software components that may be configured to process, store, and communicate information and may include one or more computer systems, data storage systems, and networking systems.

Information handling systems include auditory systems. The auditory system can include various devices and peripherals, such as speaker pucks, headsets, built in speakers, external stereo speakers, etc., allowing people to listen to music, watch audio, conduct online meetings, etc. Therefore, audio use on information handling systems has become an integral part of a person's daily routine, for work and for leisure.

Prolonged and extensive audio use and exposure has been proven to detrimental and can lead to hearing loss. High levels of audio are unsafe; however, prolonged periods at certain audio frequencies have also shown to be unsafe. As more people work remotely and conduct remote meetings, webinars, etc., and use audio resources, auditory fatigue can accumulate. Audio fatigue can be related to audio amplitude or dosage, and also to audio frequency accumulation.

### SUMMARY OF THE INVENTION

A system, method, and computer-readable medium are disclosed for auditory adaptive wellness, comprising: calibrating for user sensitivity to noise and frequency; setting a threshold for dosage fatigue (Use Case 1), a threshold for background cumulative frequency fatigue (Use Case 2), and a threshold for general frequency fatigue (Use Case 3);determining audio cumulation rates of peripherals for dosage fatigue (Use Case 1), for cumulative frequency fatigue (Use Case 2), and for general frequency fatigue (Use Case 3); calculating cumulative dosage exposure (CDE), cumulative background exposure (CBE) and cumulative frequency exposure (CFE) based on user sensitivity to noise and frequency and audio cumulation rates of the peripherals; and providing warnings and recommendations when CDE exceeds the threshold for dosage fatigue, CBE exceeds the threshold for background cumulative frequency fatigue, and CFE exceeds the threshold for general frequency fatigue.

### BRIEF DESCRIPTION OF THE DRAWINGS

The present invention may be better understood, and its numerous objects, features and advantages made apparent to those skilled in the art by referencing the accompanying drawings. The use of the same reference number throughout the several figures designates a like or similar element.

FIG. 1 is a general illustration of components of an information handling system as implemented in the system and method of the present invention;

FIG. 2 is an example of a system for adaptive auditory wellness;

FIG. 3 is a generalized flowchart for adaptive auditory wellness; and

FIG. 4 is a generalized flowchart for performing measurements for adaptive auditory wellness.

### DETAILED DESCRIPTION

A system, method, and computer-readable medium are disclosed adaptive auditory wellness. Implementations determining thresholds for audio “loudness” or dosage and audio frequency sensitivity. When thresholds are reached, compensation is applied and/or alerts provided to the user. Certain implementations provide for reducing the audio amplitude, but not reducing substantially. Implementations provide to adaptively mitigate for varying types of auditory wellness fatigue factors, such as amplitude, background and primary frequency sensitivity, etc. using a combination of alerting and user “opt-in” based time/frequency domain mitigations.

For purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, gaming, or other purposes. For example, an information handling system may be a personal computer, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a microphone, keyboard, a video display, a mouse, etc. The information handling system may also include one or more buses operable to transmit communications between the various hardware components.

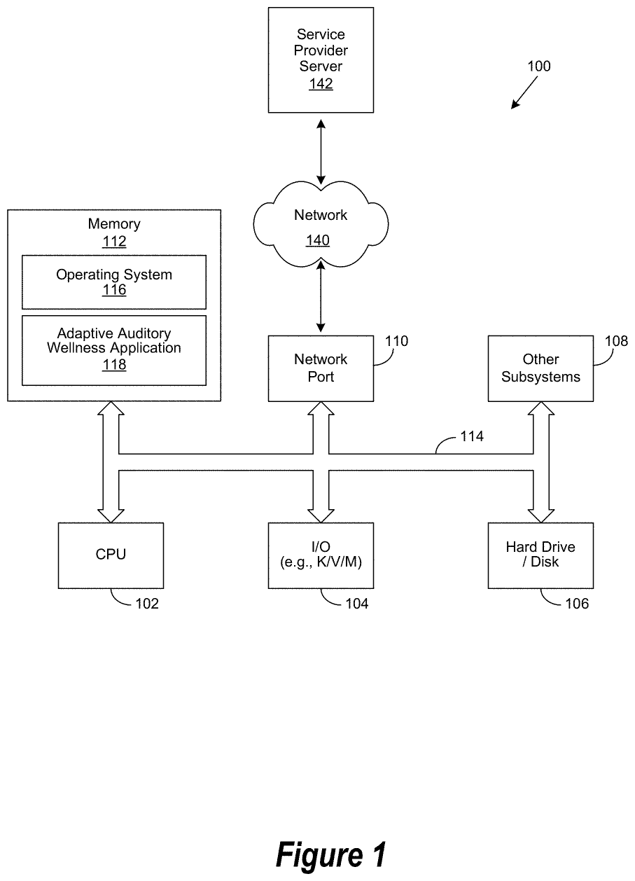

FIG. 1 is a generalized illustration of an information handling system 100 that can be used to implement the system and method of the present invention. The information handling system 100 includes a processor (e.g., central processor unit or “CPU”) 102, input/output (I/O) devices 104, such as a microphone, a keyboard, a video/display, a mouse, and associated controllers (e.g., K/V/M), a hard drive or disk storage 106, and various other subsystems 108. I/O devices 104 can include peripherals, such as speakers, headsets, and other audio output devices. Furthermore, microphones, audio sensors and the like, to receive and measure audio transmission (i.e., sound) can be included in I/O devices 104.

In various embodiments, the information handling system 100 also includes network port 110 operable to connect to a network 140, where network 140 can include one or more wired and wireless networks, including the Internet. Network 140 is likewise accessible by a service provider server 142. The information handling system 100 likewise includes system memory 112, which is interconnected to the foregoing via one or more buses 114. System memory 112 further include operating system (OS) 116. In various implementations, the system memory 112 can include an adaptive auditory wellness application 118 further described herein.

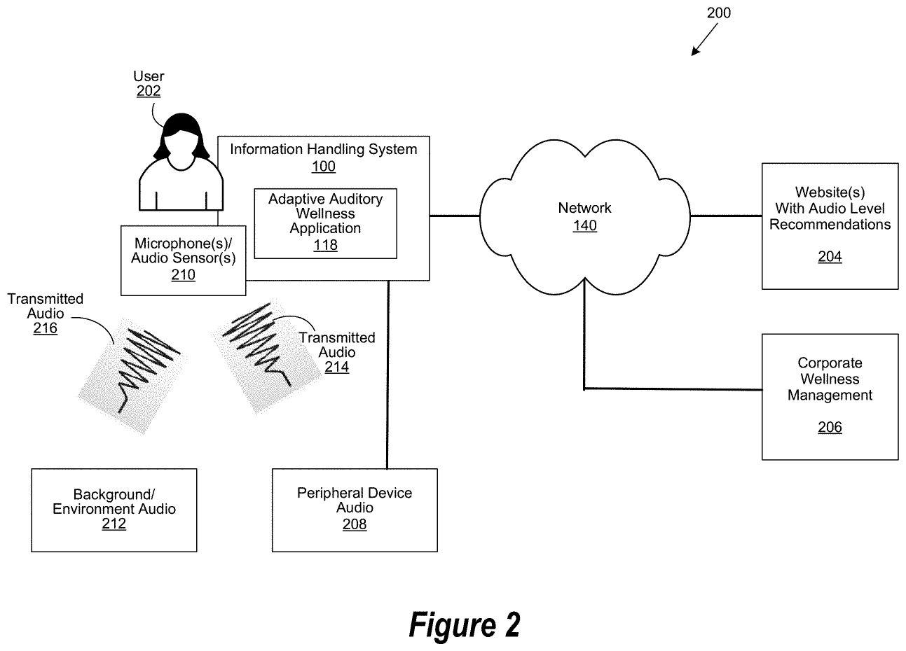

FIG. 2 shows a system for adaptive auditory wellness. The system 200 includes the information handling system 100, which includes the adaptive auditory wellness application 118. A user 202 is associated with the information handling system 100. User 202 has particular levels of use and exposure to audio.

As described above, information handling system 100 can be connected to network 140. In various implementations, information handling system 100 through network 140 communicates with one or more web sites that provide audio level recommendation 204. Examples of such websites or sources for recommendation, include public health services, such as the Centers of Disease Control and Prevention (CDC), European Union Standards for Noise, etc. For example, a recommended exposure limit (REL) is determined by the US National Institute for Occupational Safety and Health (NIOSH) for the occupational exposure limits (OELs) for noise. To reach 100% noise dose the NIOSH has determined at 85 dB(A), it would take eight hours; at 88 dB (A), it would take four hours; at 91 dB(A), it would take 2 hours; at 94 dB(A), it would take an hour; at 97 dB(A), it would take 30 minutes, and at 100 dB(A), it would take 15 minutes.

In certain implementations through the network 140, the information handling system 100 can be connected to a corporate wellness management service 206 which monitors audio level use and exposure of the user 202.

As discussed, the information handling system can include I/O devices 104 can include peripherals, such as speakers, headsets, and other audio output devices as represented by peripheral device audio 208. I/O devices 104 also can include microphones, audio sensors and the like, as represented by microphone(s)/audio sensor(s) 210.

In the system 200, there can be background/environment audio 212. Examples of background/environment audio 212 can be machine noise, children or animal noise, music from other sources, outdoor sounds, etc. Background/environment audio 212 can be any auditory source not connected to the information handling system 100 or peripheral device audio 208. Peripheral device audio 208 outputs transmitted audio 214. Background/environment audio 212 outputs transmitted audio 216.

When audio is transmitted, the audio is sent at a particular level that can correlate to a power based on Voltage Root Mean Square or Voltage RMS which is correlated to loudness. Therefore, power is correlated to loudness of a peripheral. Furthermore, frequency affects loudness, loudness varies for different frequencies. Frequency is mapped to known frequency response.

The following are three “Use Case” scenarios. “Use Case 1” is related to dosage fatigue. For example, the user 202 is working from home, and in non-stop online calls. The user 202 finds that the user 202 gets distracted, tired after using the headset of user 202 for the first three hours of the morning and have to either take a break or switch to native laptop output. “Use Case 2” is related to background cumulative frequency fatigue. For example, the user 202 is working from home, and has a reasonable (i.e., safe level) volume for online calls that are back to back. There is background noise (e.g., background/environment audio 212) from activities happening around the home of user 202, such as low frequency buzzing from contractors working inside the home. The user 202 finds that user 202 gets more fatigued than normal while in calls, even though user 202 is using the nominal volume/mixing that helps user 202 usually last through the day. “Use Case 3” is related to general frequency fatigue. For example, the user 202 is a game designer who has to listen all day to music/audio clips being designed by others, to mix into a game of user 202. The user 202 finds that for certain types of audio user 202 gets fatigued more easily before end of the day. User 202 is using nominal auditory levels throughout the day.

Implementing the system 200 and components of the system 200, the following is performed. In certain implementations, initially a calibration is performed for user 202 sensitivity to loudness and frequency, similar to known hearing tests that are performed. For example, a set of enclosed headphones with a fixed sequence of tones can be used to detect the three use cases of dosage fatigue (Use Case 1), background cumulative frequency fatigue (Use Case 2), and general frequency fatigue (Use Case 3). A threshold value is established for each use case. The threshold values can be based on recommend values, such as values from websites that provide audio level recommendation 204. T-UC1 (threshold value for Use Case 1), T-UC2 (threshold value for Use Case 1), and T-UC3 (threshold value for Use Case 1).

Different audio peripherals provide different rates of audio cumulation. For example, enclosed headsets accumulate audio faster than a laptop output speaker. Furthermore, different brands and models of peripherals have different cumulation rates. In various implementations configuration information of each peripheral in peripheral device audio 208 is taken for cumulation rate for each use case. A value is determined for each peripheral {peripheral, cumulation rate Use Case 1, cumulation rate Use Case 2, cumulation rate Use Case 3} for the peripheral device audio 208.

In certain implementations, the following is performed. In a steady state, the following are measured, where variables at set to “0” at the start of a day (e.g., work day for user 202). First, identify the peripherals of peripheral device audio 208, and begin start of audio exposure. For example, audio exposure is monitored at a start of an online call. Second, based on each of the peripherals of peripheral device audio 208, look up cumulation rates as described above, for Use Case 1, Use Case 2, and Use Case 3.

Third, for Use Case 2 (background cumulative frequency fatigue), measure background noise or background/environment audio 212 which can be measured by microphone(s)/audio sensor(s) 210. The voice of user 202 is filtered out. Fourth, for Use Case 1 (dosage fatigue), measure output loudness of the peripheral device audio 208. Fifth, for Use Case 3 (general frequency fatigue), measure overall output frequency response. Measurement can be made for example using Fast Fourier Transform (FFT), etc.

Sixth, user 202 presence is measured. In certain implementations, the use of 3D Time Of Flight (TOF) is used. If the user 202 is absent, the 3D TOF vector is set to (0,0,0). Seventh, for example using Root Mean Squared Distance (RMSE) the 3D TOF value is translated to a scalar distance or “D”, and to an overall 3D angle “A” of user 202 against a nominal plane. A value pair of {D, A} is provided. For example, going from (0,0,0) to (1,1,1), TOF=RMSE=sqrt ((1−0)\hat{ }2+(1−0)\hat{ }2+(1-0)\hat{ }2)), using 3D Pythagorean Theorem. Eighth, a normalize exposure factor as a function of 3D distance is determined. This value is called “N.” It is assumed that X ft as zero impact to audio, using trigonometry, etc.

Ninth, Cumulative Dosage Exposure (CDE) is calculated. CDE+=(cumulation rate of Use Case 1*loudness level of Use Case 1*N) minus (decay rate of Use Case 1*User Present (1) or User Absent (0)).

Tenth, Cumulative Background Exposure (CBE) is calculated. CBE+=(cumulation rate of Use Case 2*frequency level of Use Case 2*N) minus (decay rate of Use Case 2*background below threshold quietness).

Eleventh, Cumulative Frequency Exposure (CFE) is calculated. CF+=(cumulation rate of Use Case 3*frequency level of Use Case 3*N) minus (decay rate of Use Case 3*User Present (1) or User Absent (0)).

Implementations provide for Cumulative Dosage Exposure (CDE) to be compared to threshold value for Use Case 1 (T-UC1). If the threshold is reached, a trigger alert is provided to user 202. If user 202 chooses the option, a change to a different peripheral(s) (peripheral device audio 208) can follow.

Implementations provide for Cumulative Background Exposure (CBE) to be compared to threshold value Use Case 2 (T-UC2). If the threshold is reached, a warning is given to the user 202 to go to a quieter environment/room (background/environment audio 212). Other actions can also be advised to the user 202.

Implementations provide for Cumulative Frequency Exposure (CFE) to be compared to threshold value Use Case 3 (T-UC3). If the threshold is reached, if the user 202 chooses the option, frequency output is modified of the peripheral using standard post processing on audio output. In certain implementations, the level of modification can increase with the level of fatigue. It is to be noted that for T-UC3 can be a progressive sequence of thresholds for progressive set of mitigation actions.

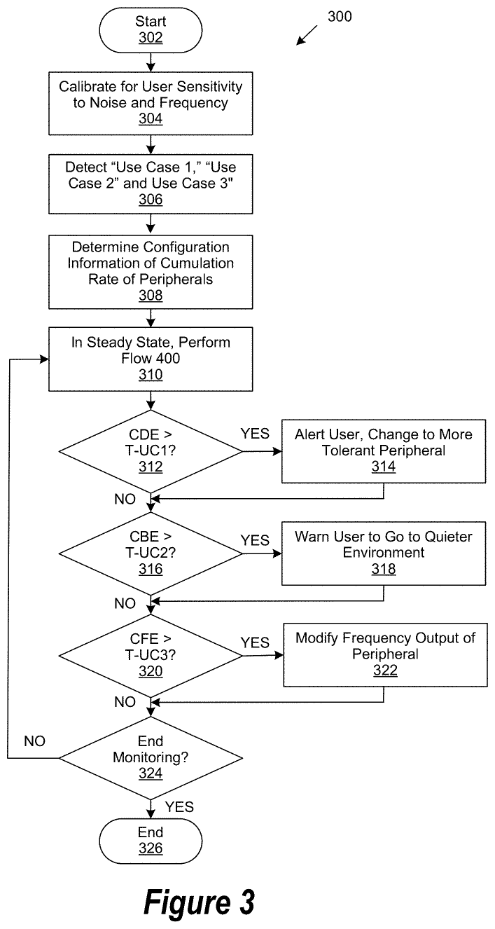

FIG. 3 is a generalized flowchart 300 for adaptive auditory wellness. In certain implementations, the process 300 is performed by the information handling system 100 described in FIG. 1 and FIG. 2. The order in which the method is described is not intended to be construed as a limitation, and any number of the described method blocks may be combined in any order to implement the method, or alternate method.

At step 302, the process 300 starts. At step 304, as discussed calibration is performed for user 202 sensitivity to (noise) loudness and frequency, similar to known hearing tests that are performed. At step 306, as discussed, detection of is performed for the example, a set of enclosed headphones with a fixed sequence of tones can be used to detect the three use cases of dosage fatigue (Use Case 1), background cumulative frequency fatigue (Use Case 2), and general frequency fatigue (Use Case 3). This can be performed using a set of enclosed headphones with a fixed sequence of tones. A threshold value is established for each use case. T-UC1 (threshold value for Use Case 1), T-UC2 (threshold value for Use Case 1), and T-UC3 (threshold value for Use Case 1).

At 308, configuration information of each peripheral in peripheral device audio 208 is taken for cumulation rate for each use case. A value is determined for each peripheral {peripheral, cumulation rate Use Case 1, cumulation rate Use Case 2, cumulation rate Use Case 3} for the peripheral device audio 208.

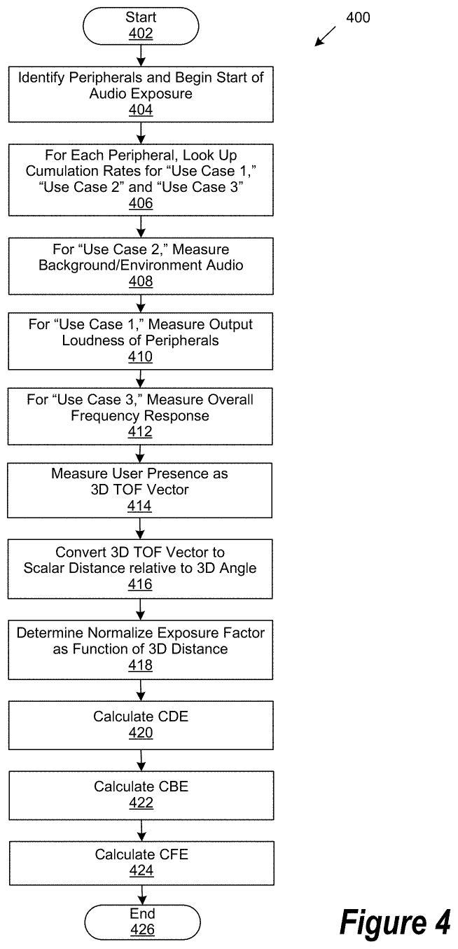

At 310, in steady state, the flowchart of FIG. 4 is performed. In a steady state, variables at set to “0” at the start of a day (e.g., work day for user 202).

Referring now to FIG. 4 which shows generalized flowchart 400 for performing measurements for adaptive auditory wellness. The order in which the method is described is not intended to be construed as a limitation, and any number of the described method blocks may be combined in any order to implement the method, or alternate method.

At step 402, the process 400 starts. At step 404, identify the peripherals of peripheral device audio 208, and begin start of audio exposure. For example, audio exposure is monitored at a start of an online call. At step 406, based on each of the peripherals of peripheral device audio 208, look up cumulation rates as described above, for Use Case 1, Use Case 2, and Use Case 3.

At step 408, for Use Case 2 (background cumulative frequency fatigue), measure background noise or background/environment audio 212 which can be measured by microphone(s)/audio sensor(s) 210. At step 410, for Use Case 1 (dosage fatigue), measure output loudness of the peripheral device audio 208. At step 412, for Use Case 3 (general frequency fatigue), measure overall output frequency response.

At step 414, measure presence of user 202. In certain implementations, the use of 3D Time Of Flight (TOF) is used. If the user 202 is absent, the 3D TOF vector is set to (0,0,0).

At step 416, the 3D TOF value is translated to a scalar distance or “D”, and to an overall 3D angle “A” of user 202 against a nominal plane. For example, Root Mean Squared Distance (RMSE) can be used. A value pair of {D, A} is provided.

At step 418, a normalize exposure factor as a function of 3D distance is determined. This value is called “N”. Trigonometry, etc. can be used, and it is assumed that X ft as zero impact to audio.

At step 420, Cumulative Dosage Exposure (CDE) is calculated. CDE+=(cumulation rate of Use Case 1*loudness level of Use Case 1*N) minus (decay rate of Use Case 1*User Present (1) or User Absent (0)).

At step 422, Cumulative Background Exposure (CBE) is calculated. CBE+=(cumulation rate of Use Case 2*frequency level of Use Case 2*N) minus (decay rate of Use Case 2*background below threshold quietness).

At step 424, Cumulative Frequency Exposure (CFE) is calculated. CFE+=(cumulation rate of Use Case 3*frequency level of Use Case 3*N) minus (decay rate of Use Case 3*User Present (1) or User Absent (0)). At step 426, the process 400 ends.

Referring back to FIG. 3. At step 312, Cumulative Dosage Exposure (CDE) is compared to threshold value for Use Case 1 (T-UC1). If CDE is greater than T-UCI, following the YES branch of step 312, at step 314, a trigger alert is provided to user 202. If user 202 chooses the option, a change to a different peripheral(s) (peripheral device audio 208) can follow.

If CDE is not greater than T-UCI, following the NO branch of step 312, at step 316, Cumulative Background Exposure (CBE) is compared to threshold value Use Case 2 (T-UC2) If the CBE is greater than T-UC2, following the YES branch of step 316, at step 318 a warning is given to the user 202 to go to a quieter environment/room (background/environment audio 212). Other actions can also be advised to the user 202.

IF CBE is not greater than T-UC2, following the NO branch of step 316, at step 320, Cumulative Frequency Exposure (CFE) is compared to threshold value Use Case 3 (T-UC3). If the CFE is greater than T-UC3,following the YES branch of step 320, at step 322, frequency output is modified of the peripheral using standard post processing on audio output. In certain implementations, the level of modification can increase with the level of fatigue. It is to be noted that for T-UC3 can be a progressive sequence of thresholds for progressive set of mitigation actions.

If CFE is not greater than T-UC3, following the NO branch of step 312, at step 324, a determination is made if monitoring is to continue. If monitoring is to continue, following the YES branch of step 324, step 310 is followed. Else, if monitoring ends, following the NO branch of step 324, at step 326, the process 300 ends.

As will be appreciated by one skilled in the art, the present invention may be embodied as a method, system, or computer program product. Accordingly, embodiments of the invention may be implemented entirely in hardware, entirely in software (including firmware, resident software, micro-code, etc.) or in an embodiment combining software and hardware. These various embodiments may all generally be referred to herein as a “circuit,” “module,” or “system.” Furthermore, the present invention may take the form of a computer program product on a computer-usable storage medium having computer-usable program code embodied in the medium.

Any suitable computer usable or computer readable medium may be utilized. The computer-usable or computer-readable medium may be, for example, but not limited to, an electronic, magnetic, optical, electromagnetic, infrared, or semiconductor system, apparatus, or device. More specific examples (a non-exhaustive list) of the computer-readable medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, or a magnetic storage device. In the context of this document, a computer-usable or computer-readable medium may be any medium that can contain, store, communicate, or transport the program for use by or in connection with the instruction execution system, apparatus, or device.

Computer program code for carrying out operations of the present invention may be written in an object oriented programming language such as Java, Smalltalk, C++ or the like. However, the computer program code for carrying out operations of the present invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Embodiments of the invention are described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The present invention is well adapted to attain the advantages mentioned as well as others inherent therein. While the present invention has been depicted, described, and is defined by reference to particular embodiments of the invention, such references do not imply a limitation on the invention, and no such limitation is to be inferred. The invention is capable of considerable modification, alteration, and equivalents in form and function, as will occur to those ordinarily skilled in the pertinent arts. The depicted and described embodiments are examples only and are not exhaustive of the scope of the invention.

Consequently, the invention is intended to be limited only by the spirit and scope of the appended claims, giving full cognizance to equivalents in all respects.

What is claimed is:

1. A computer-implementable method for auditory adaptive wellness, comprising:
calibrating for user sensitivity to noise and frequency;
setting a threshold for dosage fatigue (Use Case 1), a threshold for background cumulative frequency fatigue (Use Case 2), and a threshold for general frequency fatigue (Use Case 3);
determining audio cumulation rates of peripherals for dosage fatigue (Use Case 1), for cumulative frequency fatigue (Use Case 2), and for general frequency fatigue (Use Case 3);
calculating cumulative dosage exposure (CDE), cumulative background exposure (CBE) and cumulative frequency exposure (CFE) based on user sensitivity to noise and frequency and audio cumulation rates of the peripherals; and
providing warnings and recommendations when CDE exceeds the threshold for dosage fatigue, CBE exceeds the threshold for background cumulative frequency fatigue, and CFE exceeds the threshold for general frequency fatigue.

2. The method of claim 1, wherein audio dosage recommendations are received from one or more public health services.

3. The method of claim 1 further comprising providing audio wellness data to a corporate wellness service.

4. The method of claim 1, wherein the calibrating is performed using a fixed sequence of tones to detect for dosage fatigue (Use Case 1), cumulative frequency fatigue (Use Case 2), and for general frequency fatigue (Use Case 3).

5. The method of claim 1, wherein the warnings and recommendations include switching peripherals and/or changing environment.

6. The method of claim 1, wherein the calculating CDE, CBE, and CFE is performed at a steady state at the beginning of a work day of the user.

7. The method of claim 1, wherein calculating CDE, CBE, and CFE comprises measuring background/environment noise and user presence.

8. A system comprising:
a processor;
a data bus coupled to the processor; and
a non-transitory, computer-readable storage medium embodying computer program code, the non-transitory, computer-readable storage medium being coupled to the data bus, the computer program code interacting with a plurality of computer operations for auditory adaptive wellness and comprising instructions executable by the processor and configured for:
calibrating for user sensitivity to noise and frequency;
setting a threshold for dosage fatigue (Use Case 1), a threshold for background cumulative frequency fatigue (Use Case 2), and a threshold for general frequency fatigue (Use Case 3);
determining audio cumulation rates of peripherals for dosage fatigue (Use Case 1), for cumulative frequency fatigue (Use Case 2), and for general frequency fatigue (Use Case 3);
calculating cumulative dosage exposure (CDE), cumulative background exposure (CBE) and cumulative frequency exposure (CFE) based on user sensitivity to noise and frequency and audio cumulation rates of the peripherals; and
providing warnings and recommendations when CDE exceeds the threshold for dosage fatigue, CBE exceeds the threshold for background cumulative frequency fatigue, and CFE exceeds the threshold for general frequency fatigue.

9. The system of claim 8, wherein audio dosage recommendations are received from one or more public health services.

10. The system of claim 8 further comprising providing audio wellness data to a corporate wellness service.

11. The system of claim 8, wherein the calibrating is performed using a fixed sequence of tones to detect for dosage fatigue (Use Case 1), cumulative frequency fatigue (Use Case 2), and for general frequency fatigue (Use Case 3).

12. The system of claim 8, wherein the warnings and recommendations include switching peripherals and/or changing environment.

13. The system of claim 8, wherein the calculating CDE, CBE, and CFE is performed at a steady state at the beginning of a work day of the user.

14. The system of claim 8, wherein calculating CDE, CBE, and CFE comprises measuring background/environment noise and user presence.

15. A non-transitory, computer-readable storage medium embodying computer program code, the computer program code comprising computer executable instructions configured for:
calibrating for user sensitivity to noise and frequency;
setting a threshold for dosage fatigue (Use Case 1), a threshold for background cumulative frequency fatigue (Use Case 2), and a threshold for general frequency fatigue (Use Case 3);
determining audio cumulation rates of peripherals for dosage fatigue (Use Case 1), for cumulative frequency fatigue (Use Case 2), and for general frequency fatigue (Use Case 3);
calculating cumulative dosage exposure (CDE), cumulative background exposure (CBE) and cumulative frequency exposure (CFE) based on user sensitivity to noise and frequency and audio cumulation rates of the peripherals; and
providing warnings and recommendations when CDE exceeds the threshold for dosage fatigue, CBE exceeds the threshold for background cumulative frequency fatigue, and CFE exceeds the threshold for general frequency fatigue.

16. The non-transitory, computer-readable storage medium of claim 15, wherein audio dosage recommendations are received from one or more public health services n.

17. The non-transitory, computer-readable storage medium of claim 15, wherein the calibrating is performed using a fixed sequence of tones to detect for dosage fatigue (Use Case 1), cumulative frequency fatigue (Use Case 2), and for general frequency fatigue (Use Case 3).

18. The non-transitory, computer-readable storage medium of claim 15, wherein the warnings and recommendations include switching peripherals and/or changing environment.

19. The non-transitory, computer-readable storage medium of claim 15, wherein the calculating CDE, CBE, and CFE is performed at a steady state at the beginning of a work day of the user.

20. The non-transitory, computer-readable storage medium of claim 15, wherein calculating CDE, CBE, and CFE comprises measuring background/environment noise and user presence.
